(19) United States
(12) Patent Application Publication
(10) Pub. No.: US 2009/0247965 A1
(43) Pub. Date: Oct. 1, 2009

(54) Water Repellant Cover for Venous Access Devices

(76) Inventor: Moteah Williams, Gainesville, FL (US)

Correspondence Address: Russ Weinzimmer, 614 Nashua Street, Suite 53, Milford, NH, 03055, US

(21) Appl. No.: 12/057,344

(22) Filed: Mar. 27, 2008

(51) Int. Cl. A61M 5/00; A61F 13/00

(52) U.S. Cl. 604/263; 128/888; 128/846

(57) ABSTRACT

A waterproof, disposable, protective cover is disclosed that can completely cover a venous access device and protect it from water, for example while a patient is bathing. The cover includes a flat, adhesive outer band and an inner sheet that is able to cover the device. Superior water resistance results because no part of the protected device protrudes beyond the cover. Short-term use of the cover avoids any need to provide access to the protected device and/or expose the covered skin to air. The inner sheet can be transparent, allowing careful application and avoidance of contact between the adhesive and the device or the region of skin penetration. The disposable cover can be rectangular or square, have rounded corners, and/or be sized to protect a Groshong catheter, an intravenous access device, a Porta-cath (port) device, and/or a PICC Line. Square embodiments can measure 4 or 8 inches on a side.

### FIELD OF THE INVENTION

The invention generally relates to apparatus for attaching medical devices to the skin, and more specifically to apparatus for protecting venous access devices from exposure to water when attached to the skin.

### BACKGROUND OF THE INVENTION

Circumstances often arise in the care of a medical patient where it is desirable or necessary to frequently access the venous system of the patient, for purposes of drawing blood, hydrating the patient, administering drugs, performing blood-related procedures such as kidney dialysis, and such like. In such cases, a venous access device such as a catheter or PICC line is often installed in a vein of the patient and allowed to remain in place for an extended period of time lasting days, months, or even years. Such venous access devices typically include an insertion end that penetrates through the skin and into the vein of the patient, a valve mechanism that closes access to the vein when the device is not in use, and a connecting port that allows connection to the vein by an external device such as an IV drip source.

A certain risk of infection is always associated with long term installation of a venous access device through the skin of a patient. It is therefore necessary to keep such devices clean and as free from contaminants as possible, especially at the point of entry through the skin. The area surrounding the point of skin entry must also be kept dry, since moisture provides a breeding ground for bacteria and other disease agents. This can be a practical problem for long term installations, since the patient will eventually need to shower, bathe, or otherwise wash the affected area. At the same time, exposure to the air is important to the long term health of the skin surrounding the venous access device.

Many mechanisms are known for holding venous access devices in place on the skin of a patient. These range from simple strips of adhesive tape to much more elaborate apparatus. In most such mechanisms, a piece of tape or gauze is held securely against the point of skin penetration, and this represents a serious infection risk if the tape or gauze becomes wet.

Several types of protective coverings are known that attempt to exclude contaminants from the area surrounding a venous access device. Some of these protective coverings provide for connection to the venous access device by allowing part of the venous access device to protrude beyond the protective covering. Such protective coverings typically attempt to form a seal around the venous access device at the point where the device passes beyond the covering. However, under conditions of significant water exposure, such as when the patient takes a bath or a shower, it is very difficult to prevent some water from penetrating through the seal and reaching the point of skin penetration.

Other types of protective covering include a section that can be opened and closed so as to obtain connection to a protected venous access device. Typically, the openable and closable section includes some kind of sealing mechanism, such as a reusable adhesive coating. However, under conditions of significant water exposure, and especially after repeated opening and closing of the section and resultant degradation of the seal, it is difficult for these sealing mechanisms to prevent some water from penetrating and reaching the point of skin penetration.

Some types of protective covering are made from materials that intentionally allow the passage of air and moisture through them, thereby maintaining the health of the protected skin underneath. However, due to the nature of such materials these types of coverings cannot protect the area of skin penetration from becoming wet under conditions of significant water exposure.

### SUMMARY OF THE INVENTION

A waterproof, disposable, protective cover is disclosed that can be temporarily applied to the skin of a patient so as to completely cover a venous access device, for example while the patient is bathing or otherwise exposed to water. Because the invention completely covers the venous access device and skin penetration area, it provides superior water resistance. Because it is disposable and used only for short periods, the invention avoids the need to provide for connection to the venous access device, and it avoids the need to provide for exposure of covered skin to the air. In one aspect of the invention, the center of the cover is transparent, so as to allow a user to more carefully apply the cover and avoid any possibility of contact between the adhesive outer area of the cover and the venous access device and/or region of skin penetration.

The protective cover includes a flat band that is able to completely surround the venous access device when the venous access device is attached to the patient, the flat band being made of water repellant material that is able to adhesively attach to the skin of the patient. The protective cover further includes a flexible, water repellant cover sheet that makes a continuous, water repellant seal with the flat band and is able to cover the venous access device when the flat band surrounds the venous access device and is adhesively attached to the patient's skin. In this way, the water repellant cover sheet together with the flat adhesive band form a protective cover that inhibits exposure of the venous access device to water. As mentioned above, in one aspect of the invention the cover sheet is transparent.

In preferred embodiments, the disposable cover is able to completely cover and protect of a Groshong catheter, an intravenous access device, a Porta-cath (port) venous access device, and/or a PICC Line. In some preferred embodiments the disposable cover is rectangular or square in shape, and in some of these preferred embodiments the corners of the rectangle are rounded. In some embodiments where the disposable cover is rectangular or square the dimensions of the disposable cover are approximately 4 inches by 4 inches, and in other embodiments where the disposable cover is rectangular or square the dimensions of the disposable cover are approximately 8 inches by 8 inches.

### BRIEF DESCRIPTION OF THE DRAWINGS

FIG. 1 is a diagram of an embodiment of the invention that is square with rounded corners;

FIG. 2 is a perspective drawing from above of a rectangular embodiment with rounded corners covering an intravenous access device installed on the arm of a patient; and

FIG. 3 is a perspective drawing from the side of the embodiment of FIG. 2 covering a Groshong catheter inserted into the arm of a patient.

### DETAILED DESCRIPTION OF PREFERRED EMBODIMENTS

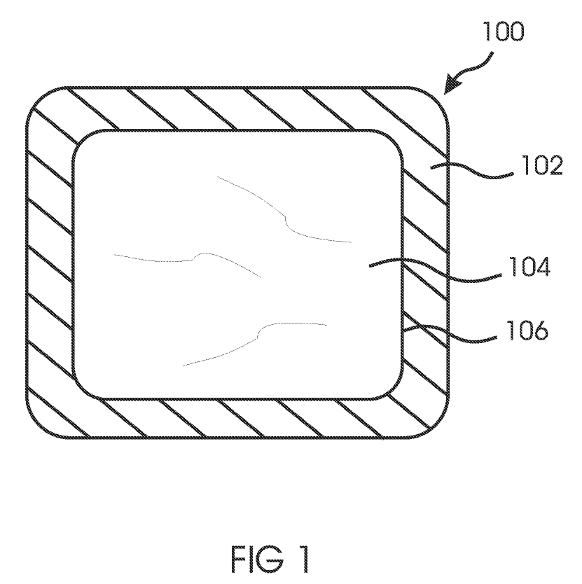

With reference to FIG. 1, the disposable cover 100 of the present invention includes a flat band 102 that is able to completely surround a venous access device when the venous access device is attached to a patient. The flat band 102 is made of water repellant material and is able to adhesively attach to the skin of the patient. A flexible, water repellant cover sheet 104 fills the space surrounded by the flat band 102 and makes a continuous, water repellant seal 106 with the flat band 102. The cover sheet 104 is large enough to cover the venous access device when the flat band 102 is adhesively attached to the patient's skin, and forms water repellant cover together with the flat band 102 that inhibits exposure of the venous access device to water. In the embodiment of FIG. 1, the disposable cover is approximately square in shape, with rounded corners.

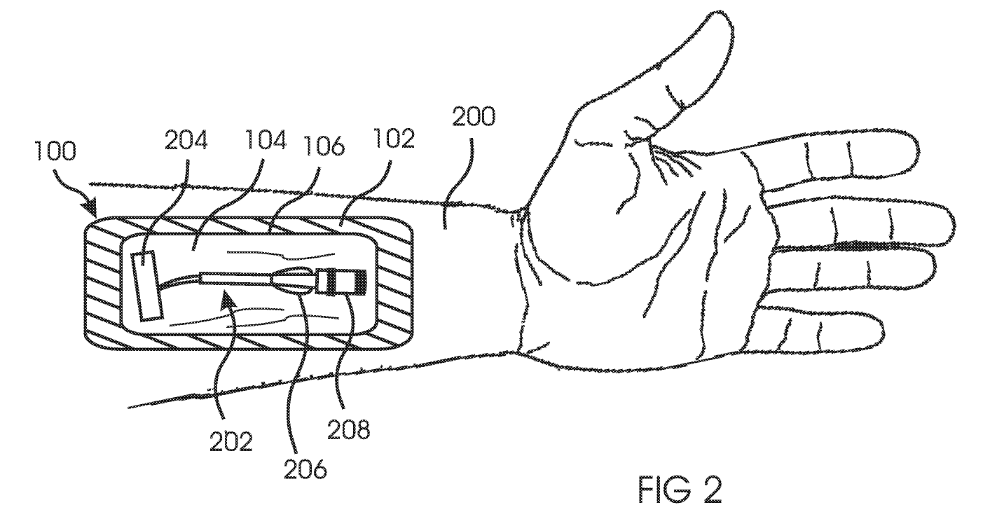

FIG. 2 is a perspective view of a preferred embodiment 100 that has been adhesively attached to the arm 200 of a patient, such that it completely covers a venous access device 202 installed in the patient's arm 200. In this embodiment the disposable cover 100 is rectangular with rounded corners, and the cover sheet 104 is transparent. This allows a user to continuously observe the exact location and configuration of the venous access device 202 as the disposable cover 100 is being adhesively attached to the patents skin, thereby avoiding accidental contact between the venous access device 202 and the adhesive flat band 102 that surrounds the disposable cover 100. Note that the disposable cover 100 completely covers the venous access device 202, including the tape 204 covering the area of skin penetration, the valve 206 that blocks access to the vein when the venous access device 202 is not in use, and the connecting port 208 that provides for connection to the patient's vein. In various preferred embodiments, the disposable cover 100 is able to completely enclose virtually any apparatus associated with the venous access device 202, including attachment mechanisms and protective coverings that exclude contaminants and are intended for longer term use.

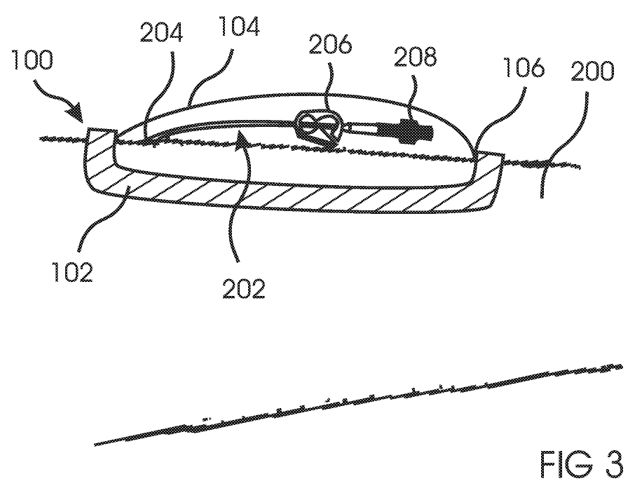

FIG. 3 is a perspective view from the side of the embodiment of FIG. 2. In FIG. 3 the disposable cover 100 is protecting a Groshong catheter 202. It can be seen from the figure that the cover sheet 104 of the disposable cover 100 is sufficiently large to extend above and over the Groshong catheter 202 when the adhesive outer band 102 of the disposable cover 100 is surrounding the catheter 202 and firmly attached to the skin of the patient's arm 200.

Other modifications and implementations will occur to those skilled in the art without departing from the spirit and the scope of the invention as claimed. Accordingly, the above description is not intended to limit the invention except as indicated in the following claims.

What is claimed is:

1. A waterproof, transparent, disposable cover for preventing exposure of a venous access device to water when the venous access device is attached to a patient, the disposable cover comprising:
a flat band that is able to completely surround the venous access device when the venous access device is attached to the patient, the flat band being made of water repellant material and being able to adhesively attach to the skin of the patient; and
a flexible, transparent, water repellant cover sheet that makes a continuous, water repellant seal with the flat band and is able to cover the venous access device when the flat band surrounds the venous access device and is adhesively attached to the patient's skin, the water repellant cover sheet thereby combining with the flat band to form a cover that inhibits exposure of the venous access device to water.

2. The disposable cover of claim 1, wherein the disposable cover is able to prevent exposure of a Groshong catheter to water.

3. The disposable cover of claim 1, wherein the disposable cover is able to prevent exposure of an intravenous access device to water.

4. The disposable cover of claim 1, wherein the disposable cover is able to prevent exposure of a Porta-cath (port) venous access device to water.

5. The disposable cover of claim 1, wherein the disposable cover is able to prevent exposure of a PICC Line venous access device to water.

6. The disposable cover of claim 1, wherein the disposable cover is one of rectangular and square in shape.

7. The disposable cover of claim 6, wherein the corners of the disposable cover are rounded.

8. The disposable cover of claim 6, wherein the dimensions of the disposable cover are approximately 4 inches by 4 inches.

9. The disposable cover of claim 6, wherein the dimensions of the disposable cover are approximately 8 inches by 8 inches.

10. A waterproof, disposable cover for preventing exposure of a venous access device to water when the venous access device is attached to a patient, the disposable cover comprising:
a flat band that is able to completely surround the venous access device when the venous access device is attached to the patient, the flat band being made of water repellant material and being able to adhesively attach to the skin of the patient; and
a flexible, water repellant cover sheet that makes a continuous, water repellant seal with the flat band and is able to cover the venous access device when the flat band surrounds the venous access device and is adhesively attached to the patient's skin, the water repellant cover sheet thereby combining with the flat band to form a cover that inhibits exposure of the venous access device to water.

11. The disposable cover of claim 10, wherein the disposable cover is able to prevent exposure of a Groshong catheter to water.

12. The disposable cover of claim 10, wherein the disposable cover is able to prevent exposure of an intravenous access device to water.

13. The disposable cover of claim 10, wherein the disposable cover is able to prevent exposure of a Porta-cath (port) venous access device to water.

14. The disposable cover of claim 10, wherein the disposable cover is able to prevent exposure of a PICC Line venous access device to water.

15. The disposable cover of claim 10, wherein the disposable cover is one of rectangular and square in shape.

16. The disposable cover of claim 15, wherein the corners of the disposable cover are rounded.

17. The disposable cover of claim 15, wherein the dimensions of the disposable cover are approximately 4 inches by 4 inches.

18. The disposable cover of claim 15, wherein the dimensions of the disposable cover are approximately 8 inches by 8 inches.
